(19) United States
(12) Patent Application Publication
(10) Pub. No.: US 2022/0084634 A1
(43) Pub. Date: Mar. 17, 2022

(54) PORTABLE SPECIMEN COLLECTION AND TESTING KIT

(71) Applicant: Labware Holdings, Inc., Wilmington, DE (US)

(72) Inventors: Vance V. KERSHNER, Wilmington, DE (US); Jon F. Raiford, Newark, DE (US); Eilze Reinecke, Chadds Ford, PA (US); Vaughn Hardin, Wilmington, DE (US); Peter Payne, Wilmington, DE (US); John Peet IV, Wilmington, DE (US); Russell Robertson, Chadds Ford, PA (US); Meric Overman, Chadds Ford, PA (US); Edward Krasovec, Media, PA (US)

(73) Assignee: Labware Holdings, Inc., Wilmington, DE (US)

(21) Appl. No.: 17/021,818

(22) Filed: Sep. 15, 2020

(51) Int. Cl. G16H 10/40; G16H 10/60; G06Q 30/00; G16H 40/20; G06Q 50/26; G06K 7/14; A61B 10/00

(52) U.S. Cl. CPC G16H 10/40; G16H 10/60; G06Q 30/0185; G16H 10/20; G06Q 50/265; G06K 7/1413; A61B 10/0096; G16H 40/20

(57) ABSTRACT

In one embodiment, a portable specimen and collection kit is provided. The kit provides improved patient triage and streamlined specimen collection with a cloud-based, HIPAA-compliant and user-friendly software that is accessed by a field operations person via a tablet computer and a mobile hotspot. The kit may use a software platform currently deployed in 7 U.S. state public health labs and most of the public health labs in Canada. Once patient demographic details and answers to triage questions are captured, labels are printed on an included wireless portable label printer and affixed to the specimen container(s). The software routes the specimen to the appropriate location (public health or commercial lab) for testing based on priority and workload. The order information is transmitted electronically to the testing laboratory information system so that the specimen is immediately ready to be tested upon receipt in the laboratory.

### BACKGROUND

The are many difficulties associated with current methods for testing for infectious diseases such as COVID-19. These difficulties include: a general shortage of facilities capable of running COVID-19 tests; a general shortage of testing kits; limitations in the ability to triage so that the most critical cases can be routed to enable testing in the shortest possible time; patient interview and specimen collection processes that rely on handwritten records which lead to transcription errors; a lack of a robust point of care COVID-19 testing capability; and general delays in the communication of COVID-19 test results once available.

### SUMMARY

In one embodiment, a portable specimen and collection kit is provided. The kit provides improved patient triage and streamlined specimen collection with a cloud-based, HIPAA-compliant and user-friendly software that is accessed by a field operations person via a tablet computer and a mobile hotspot. The kit uses a software platform currently deployed in U.S. state public health labs and Canadian public health labs. Once patient demographic details and answers to triage questions are captured, labels are printed on an included wireless portable label printer and affixed to the specimen container(s). The software then routes the specimen to the appropriate location (public health or commercial lab) for testing based on priority and workload. The order information is transmitted electronically to the testing laboratory information system so that the specimen is immediately ready to be tested upon receipt in the laboratory.

In another embodiment, the kit may further include a portable molecular analyzer that allows a variety of tests to be performed in the field. A field operator may use the tablet computer included in the kit to register the patient and produce a specimen label using the included printer. The highest priority samples may be tested on the molecular analyzer at the time of collection. The results of the test may be electronically transmitted, recorded, and subsequently forwarded to relevant government agencies via the included mobile hotspot. Total turnaround time from a patient arriving at a mobile collection site to reported results may be less than 45 minutes. If appropriate, lower priority samples can be collected and routed to either a government or private testing facility. The order information may be transmitted electronically to the testing facility so that the specimen is immediately ready to be tested upon receipt by the lab, eliminating manual data entry and the 2-5% transcription errors that go with it.

The described portable specimen and collection kit also provides support for call center operations to allow patients to schedule appointments for specimen collection. The kit is extensible and can be used for the field testing of other diseases such as tuberculosis, Zika, sexually transmitted diseases, and Ebola.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is an illustration of an exemplary environment for the use of a portable specimen collection and testing kit;

FIG. 2 is an illustration of an exemplary portable specimen collection and testing kit;

FIG. 3 is an illustration of a first or top level of a portable specimen collection and testing kit;

FIG. 4 is an illustration of a second or bottom level of a portable specimen collection and testing kit;

FIG. 5 is an illustration of a method for collecting and testing a specimen by a portable specimen collection and testing kit;

FIG. 6 is an illustration of a method for collecting a specimen by a portable specimen collection and testing kit;

FIG. 7 is an illustration of a method for collecting and testing a specimen by a portable specimen collection and testing kit; and

FIG. 8 shows an exemplary computing environment in which example embodiments and aspects may be implemented.

### DETAILED DESCRIPTION

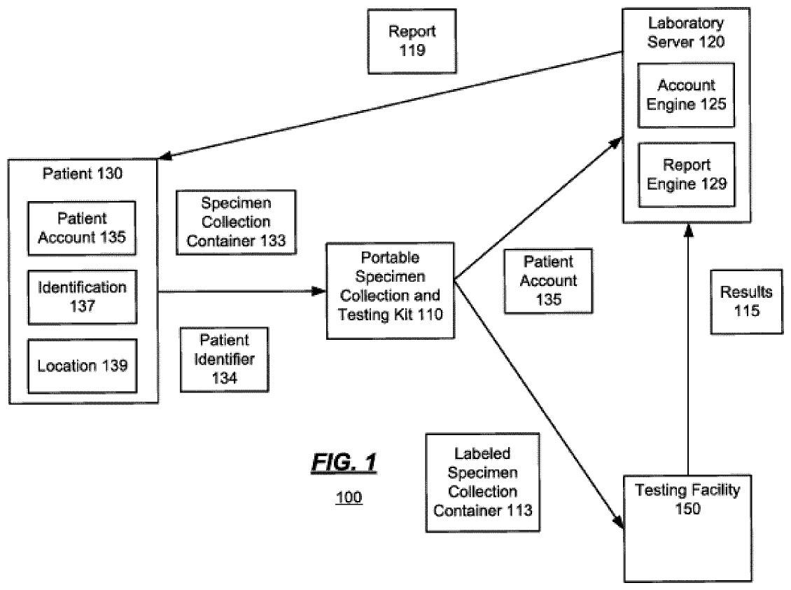

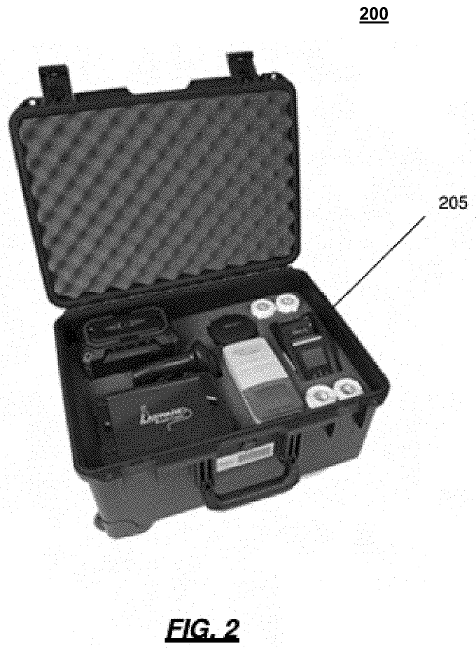

FIG. 1 is an illustration of an exemplary environment 100 for the use of a portable specimen collection and testing (PSCT) kit 110. The PSCT kit 110 may include a case or housing that is adapted to store a plurality of components such as a scanner, a label printer, a tablet computer, a Wi-Fi hotspot, and a specimen collection kit. Depending on the embodiment, the PSCT kit 110 may further include a molecular analyzer that can perform one or more tests on a collected specimen. Suitable examples of PSCT kits 110 are described further with respect to FIGS. 2-4.

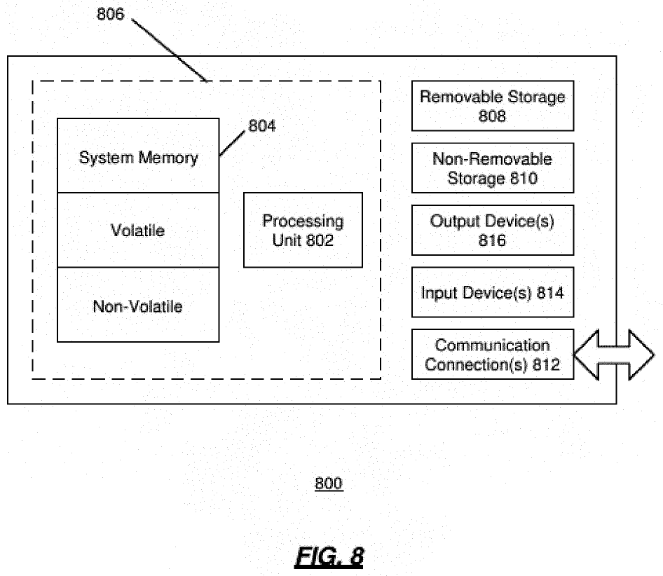

The PSCT kit 110 may be carried by an operator or user to a location 139 associated with a patient 130. Depending on the embodiment, the patient 130 may have made an appointment to be tested by the PSCT kit 110 through a laboratory server 120 using a telephone or a computer-based appointment system. The laboratory server 120 may be a cloud-based server and may be implemented using one or more computing devices such as the computing device 800 illustrated with respect to FIG. 8.

The PSCT kit 110 may be used to test for a variety of diseases and/or conditions such as tuberculosis, Zika, sexually transmitted diseases, COVID-19, and Ebola. The PSCT kit 110 may also be used to perform drug testing and paternity testing. It is contemplated that the PSCT kit 110 may be used to perform any type of specimen-based tests on patients 130.

With reference to FIG. 2 is shown an exemplary PSCT kit 110. In the example shown, the PSCT kit 110 includes a case 205 that is adapted to store a plurality of components. The case 205 may be made of a variety of lightweight and durable materials to allow users to easily carry the PSCT kit 110 to a variety of locations and environments. Suitable materials include plastic and aluminum. Other materials may be used.

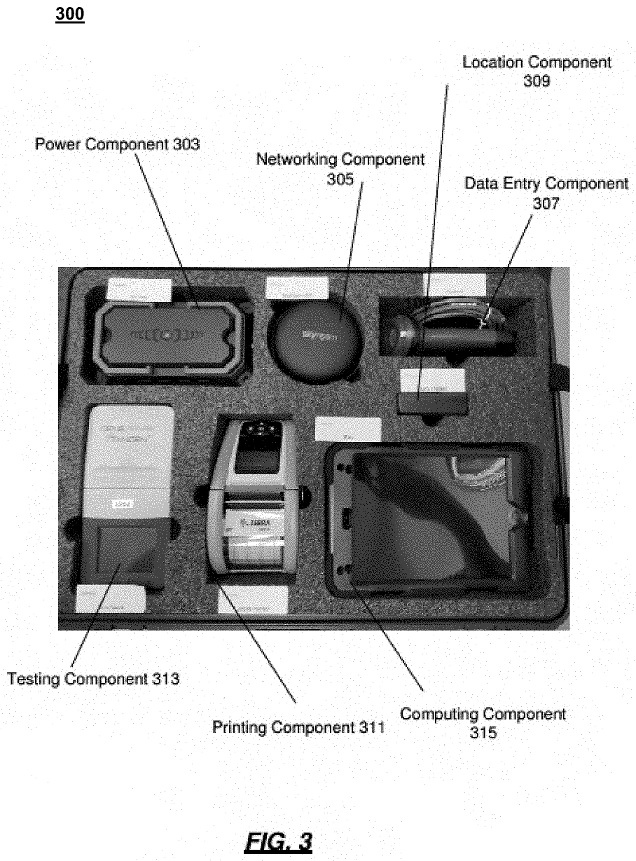

The PSCT kit 110 may include a plurality of components arranged into plurality of levels. FIG. 3 is an illustration of a first or top level of a PSCT kit 110. The components of the top level include a power component 303, a networking component 305, a data entry component 307, a location component 309, a printing component 311, a testing component 313, and a computing component 315. More or fewer components may be supported.

The power component 303 may provide power to one or more of the other components of the PSCT kit 110. Depending on the embodiment, the power component 303 may be a rechargeable (or non-rechargeable) battery or a series of batteries. Any type of batteries may be supported. The power component 303 may allow the PSCT kit 110 to be used at a variety of locations including locations where power may not be available.

The networking component 305 may create a networking connection that the various components of the PSCT kit 110 may use to communicate with one or more external computing devices such as the laboratory server 120 of FIG. 1. In some embodiments, the networking component 305 may be a Wi-Fi hotspot that creates a Wi-Fi network that can be used by the various components of the PSCT kit 110 to connect to the Internet via a cellular connection. Other types of networks and networking technologies may be used.

The data entry component 307 may be a scanner or camera that may be used by an operator of the PSCT kit 110 to scan or capture data that may be used to identify a patient 130. For example, the data entry component 307 may be used to scan or capture an image of a driver's license of a patient 130, may be used to capture an image of a face of the patient 130, or may be used to capture a fingerprint of the patient 130. The captured images or scans may be used to authenticate the patient 130, to determine a patient account 135 associated with the patient 130, and to automatically extract information about the patient 130 (e.g., name and address). By automatically extracting information about the patient 130 from a driver's license user error associated with manually entering data is greatly reduced.

The location component 309 may be a GPS, or other device, that is capable of determining a current location 139 of the PSCT kit 110. The determined location 139 may be used to associate the location 139 with any testing performed by the PSCT kit 110 or may be used to recover the PSCT kit 110 in the event that the PSCT kit 110 is stolen or misplaced.

The printing component 311 may be portable printer that is configured to print one or more labels that can be affixed to items such as specimen collection containers and envelopes. Any type of label printer may be used.

The testing component 313 may be a device that is capable of performing one or more tests on a collected specimen, such as a molecular analyzer. A suitable molecular analyzer is the Tangen GeneSpark™ analyzer. Other molecular analyzers may be used.

The computing component 315 may be a tablet computer or any computing device that is capable of interfacing with some or all of the above described components of the PSCT kit 110. The computing component 315 may control the data entry component 307, may communicate with the laboratory server 120 via the networking component 305, may control the printing component 311, and may control the testing component 313. Depending on the embodiment, the computing component 315 may interface with the various components of the PSCT kit 110 via the networking component 305 or through another networking interface or protocol such as Bluetooth. A suitable computing component 315 is the computing device 800 illustrated with respect to FIG. 8.

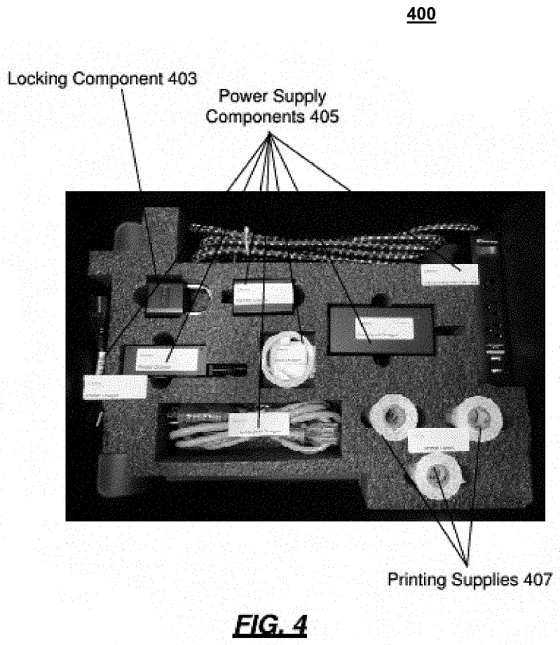

FIG. 4 is an illustration of a second or bottom level of a PSCT kit 110. More layers may be included. The components of the bottom level include a locking component 403, one or more power supply components 405, and printing supplies 407. Depending on the embodiment, the bottom level may further include items such as mailing envelopes and one or more specimen collection containers. More or fewer components may be supported.

The locking component 403 may include one or more locks that may be used by the operator of the PSCT kit 110 when not in use. The locking component 403 may include means to keep the PSCT kit 110 from being open, as well as means for securing the PSCT kit 110 to an object so that the PSCT kit 110 cannot be stolen. Any type of lock or locking systems may be used.

The one or more power supply components 405 may include power supplies that may be used to charge, or supply power to, one or more of the power component 303, the networking component 305, the data entry component 307, the printing component 311, the testing component 313, and the computing component 315. Depending on the embodiment, the power supply components 405 may allow some or all of the components to connect and receive power from the power component 303 and/or a mains power source that may be accessible at a current location of the PSCT kit 110.

The one or more printing supplies 407 may include supplies that may be used by the printing component 311 including labels, paper, and toner. Other types of printing supplies may be included.

Returning to FIG. 1, when the PSCT kit 110 arrives at a location 139 associated with the patient 130, the user or operator of the PSCT kit 110 may retrieve a computing device such as a tablet computer (i.e., the computing component 311) from the PSCT kit 110. The user may further activate the networking component 305, such as a Wi-Fi hotspot, that may allow the tablet computer to communicate (via the Internet) with the laboratory server 120. Depending on the embodiment, the tablet computer may communicate with other components of the PSCT kit 110 (e.g., a scanner, a printer, a GPS, and optionally a molecular analyzer), via a Wi-Fi network created by the Wi-Fi hotspot. Alternatively, or additionally, the PSCT kit 110 may communicate with the other components using a networking protocol such as Bluetooth, or through a wired networking connection (e.g., USB or ethernet).

The tablet computer associated with the PSCT kit 110 may display an instruction to the user to verify the identity of the patient 130. In some embodiments, the user may verify the identity of the patient 130 by requesting an identification 137 of the patient 130. The identification 137 may include drivers' licenses and passports, for example. Other types of identification 137 may be supported.

In some embodiments, the user may use a scanner of the data entry component 307 to scan a barcode or other code on the identification 137. The scanner may retrieve a patient identifier 134 from the barcode and may provide the patient identifier 134 to the tablet computer. The patient identifier 134 may include the name and address of the patient 130, for example.

In other embodiments, the user may capture an image of the identification 137 (e.g., using the scanner or a camera associated with the tablet computer), and the tablet computer may use OCR to extract the patient identifier 134 (e.g., the name and address) from the identification 137.

The PSCT kit 110 may use the patient identifier 134 to determine a patient account 135 that corresponds to the patient identifier 134. In some embodiments, the PSCT kit 110 may determine the patient account 135 by comparing the patient identifier 134 with stored patient accounts 135 or one or more patient accounts 135 that are scheduled for testing. Alternatively, or additionally, the PSCT kit 110 may provide the patient identifier 134 to an account engine 125 of the laboratory server 120, and the account engine 125 may provide a matching patient account 135 to the PSCT kit 110.

To ensure that the data associated with a patient account 135 is entered correctly, the patient 130 may have created a patient account 135 using a personal computing device prior to visiting the PSCT kit 110. The patient 130 may have entered information such as their name, address, and insurance information into an electronic form provided by the account engine 125 of the laboratory server 120.

If no matching patient account 135 is determined by either the PSCT kit 110 or the account engine 125, the PSCT kit 110 may prompt the user to create a patient account 135 for the patient 130. The patient account 135 may be automatically pre-populated using the patient identifier 134 scanned from the identification 137. For example, the tablet computer may display instructions to the user to confirm information such the name and address of the patient 130, instructions to ask the patient 130 for their insurance information, and instructions to ask the patient 130 for contact information such as an email address or telephone number. The collected information may then be transmitted from the PSCT kit 110 to the account engine 125 and may be used to create a patient account 135 for the patient 130.

If a matching patient account 135 is determined, the user of the PSCT kit 110 may be instructed to have the patient 130 confirm the information in the patient account 135. For example, the information may be displayed on the tablet computer and shown to the patient 130. Any updates to the information may be entered and provided to the account engine 125 of the laboratory server 120.

After verification, the PSCT kit 110 may print one or more labels corresponding to the particular test that is being performed. The labels may include information such as the name of the patient 130, and an identifier of the test being performed. Each label may also include a barcode, or other computer-readable code, that may identify the associated patient account 135. The one or more labels may be printed by the printing component 311 of the PSCT kit 110. Each label may also include the location 139 of the PSCT kit 110. The location 139 may be determined by the location component 309.

After the labels are printed, the PSCT kit 110 may instruct the user to affix the labels to a specimen collection container 133. The specimen collection container 133 with the affixed label is referred to herein as the labeled specimen collection container 113. Examples of a specimen collection container 133 include test tubes and specimen collection cups, for example. Depending on the embodiment, the specimen collection container 133 may be part of, or separate from, the PSCT kit 110.

The PSCT kit 110 may instruct the user of the PSCT kit 110 to collect a specimen from the patient 130, and to place the collected specimen in the labeled specimen collection container 113. The type of specimen that is collected may depend on the particular test being performed by the PSCT kit 110.

After the specimen is secured in the the labeled specimen collection container 113, the PSCT kit 110 may determine a testing facility 150 that will test the specimen. The testing facility 150 may be a government-based testing facility 150 or may be a private testing facility 150. In some embodiments, the laboratory server 120 may determine what testing facility 150 to use based on a variety of factors such as the insurance associated with the patient 130 (e.g., some insurance providers may contract with different testing facilities), the type of test being performed (e.g., some testing facilities 150 may only offer certain tests), the urgency or need for the test (e.g., some testing facilities 150 may offer faster or expedited testing), the location 139 of the PSCT kit 110 and the locations of the testing facilities 150 (e.g., closer testing facilities 150 may be preferred), and the overall busyness of the each testing facility 150 (e.g., less busy testing facilities may be favored).

In some embodiments, the testing facility 150 may be selected by the laboratory server 120. Alternatively, the testing facility 150 may be selected by the PSCT kit 110. After the testing facility is selected, the PSCT kit 110 may instruct the user to mail, or otherwise provide, the labeled specimen collection container 113 to the testing facility 150. Depending on the embodiment, the printing component 311 of the PSCT kit 110 may print a label with the address of the selected testing facility 150 that may be affixed to the box or envelope that is used to mail the labeled specimen collection container 113. An electronic record of the test and associated patient account 135 may be provided to the testing facility 150 so that the testing facility 150 may begin processing the specimen as soon as it is received.

When the labeled specimen container 113 is received at the testing facility 150, a technician (or other user) may scan the barcode on the label that was generated by the PSCT kit 110. The barcode may identify the associated patient account 135 to the testing facility 150, and a record that the labeled specimen container 113 was received may be generated and provided to the laboratory server 120.

After the testing facility 150 completes the testing, the results 115 and an indication of the associated patient account 135 may be provided to the laboratory server 120. The laboratory server 120 may record the results 115 and the account engine 125 may associate the results 115 with the patient account 135.

The report engine 129 of the laboratory server 120 may generate a report 119 that is provided to the patient 130. The report 119 may include information such as the name of the patient 130, an identifier of the associated patient account 135, and the results 115 of the test. Other information may be included in the report 119. The report 119 may be mailed or electronically provided to the patient 130 based on preferences in the patient account 135. For example, the report engine 129 may send an electronic message (e.g., email or text) to the patient 130. The electronic message may include a URL that the patient 130 may use to view the report or to download the report 119 to a computing device of their choosing.

In some embodiments, a report 119 may also be provided to a physician or insurance company associated with the patient 130. In addition, depending on the disease or condition that was tested, a report 119 may also be provided to one or more local, state, or federal agencies that are tasked with tracking the particular disease or condition. In such cases any information that may be used to identify the patient 130 may be removed from the report 119 before it is provided.

As described above, the PSCT kit 110 may further include a testing component 313 (e.g., a molecular analyzer). In some embodiments, after a specimen is collected by a user from a patient 130, the tablet computer may instruct the user to place the specimen into the molecular analyzer for testing. After the molecular analyzer generates results 115, the results 115 may be provided by the PSCT kit 110 to the account engine 125 of the laboratory server 120 as described above.

In addition, even though the specimen was tested by the molecular analyzer, in some embodiments the user may be instructed by the PSCT kit 110 to place the specimen in the labeled specimen collection container 113. The labeled specimen collection container 113 may then be provided and tested by a testing facility 150 as described above. The report 119 generated for the patient account 135 may include both the results 115 generated by the molecular analyzer and the results generated by the testing facility 150.

In some embodiments, the molecular analyzer may be used to test every patient 130 visited by the PSCT kit 110. Alternatively, only a subset of the patients 130 visited by the PSCT kit 110 may be tested using the molecular analyzer. Whether or not a patient 130 is tested using the molecular analyzer or just the testing facility 150 may be determined based on a variety of factors including the urgency of the test being performed, a status or priority associated with the patient 130, and the type of insurance associated with the patient 130. Other factors may be considered. Whether or not a patient 130 is tested using the molecular analyzer may be determined by the PSCT kit 110 and/or the laboratory server 120.

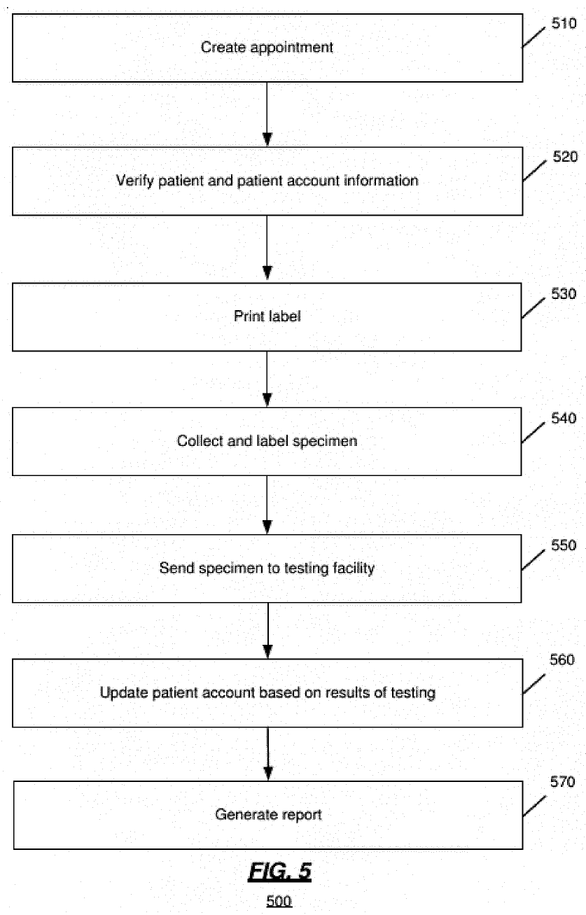

FIG. 5 is an illustration of a method 500 for testing a specimen by a PSCT kit 110. The method 500 may be performed by one or more of the PSCT kit 110 and the laboratory server 120.

At 510, an appointment is created. A patient 130 may contact the laboratory server 120 via the Internet (or telephone) to schedule an appointment. The appointment may be for an appointment to be tested by the PSCT kit 110. The test may be for a variety diseases and/or health conditions such as COVID-19. The patient 130 may provide their patient account 135 and a desired location 139 for the test. If the patient 130 does not already have a patient account 135 with the laboratory server 120, the account engine 125 may create a patient account 135 for the patient 130. The patient account 135 may include information such as name, date of birth, address, and insurance information. Other information may be included.

At 520, the patient and patient account information is verified. The patient 130 may be verified by the PSCT testing kit 110. With respect to the patient 130, the PSCT kit 110 may verify the patient by extracting a patient identifier 134 from identification 137 associated with the patient 130. For example, the user of the PSCT kit 110 may scan a driver's license associated with the patient 130 and may extract the patient identifier 134 from the driver's license. If the patient identifier 134 matches the patient account 135 associated with the patient 130, then the patient 130 is verified.

With respect to the account information, after verifying the patient 130, the PSCT kit 110 may display account information from the patient account 135 to the patient 130 and may ask the patient 130 to verify that the information is correct. The information may include name, address, contact information, and insurance information. Other information may be included. Depending on the embodiment, the patient 130 may verify the information, or may update the information using the tablet computer of the PSCT kit 110.

At 530, a label is printed. The label may be printed by a printer contained in the PSCT kit 110. Depending on the embodiment, the label may indicate the test that is being performed, may indicate a location 139 associated with the patient 130, and may indicate the patient account 135 associated with the patient 130. For example, the label may include a barcode that identifies the patient account 135. After the label is printed the user of the PSCT kit 110 may affix the label to a specimen collection container 133.

At 540, the specimen is collected and labeled. The user may collect the specimen from the patient 130 using a specimen collector provided in the PSCT kit 110. After collecting the specimen, the user may place the specimen in the labeled specimen collection container 113. Any method for collecting a specimen may be used.

At 550, the specimen is sent to a testing facility. The specimen in the labeled specimen collection container 113 may sent by the user of the PSCT kit 110. In some embodiments, the labeled specimen collection container 113 may be mailed to a testing facility 150 at an address printed on the label. The particular testing facility 150 may have been selected by the user or the laboratory server 120 based on a variety of factors such as the type of test being performed, a priority associated with the test and/or the patient 130, and the location 139 of the patient 130.

At 560, the patient account is updated based on results of the testing. The patient account 130 may be updated by the account engine 125 of the laboratory server 120. The testing facility 150 may receive the labeled collection container 113 and may scan the label to determine the patient account 135 associated with the test. After the test is performed on the specimen, the testing facility 150 may send results 115 of the test along with an identifier of the patient account 135 to the account engine 125 of the laboratory server 120. The account engine 125 may then store the results 115 with the patient account 135.

At 570, a report is generated. The report 119 may be generated by the report engine 129 of the laboratory server 120. The report 119 may include information such as the results 115 of the particular test that was performed. The report 119 may further include an identifier of the patient 130 and/or the patient account 135. Depending on the embodiment, the report 119 may be provided to the patient 130, an insurance company associated with the patient 130, an employer of the patient 130, and a doctor associated with the patient 130. In addition, the report 119 may be provided to one or more local, state, or federal agencies who may be tracking or monitoring a particular disease or condition that was tested for. Where the report 119 is provided to a third-party any information that may be used to identify the associated patient 130 may be removed or redacted from the report 119.

In some embodiments, the report engine 129 of the laboratory server 120 may send an electronic message (e.g., email or text) to the patient 130. The electronic message may include a URL, or link, to the report 119. The patient 130 may then use the link to view the report or to download the report 119 to a computing device of their choosing.

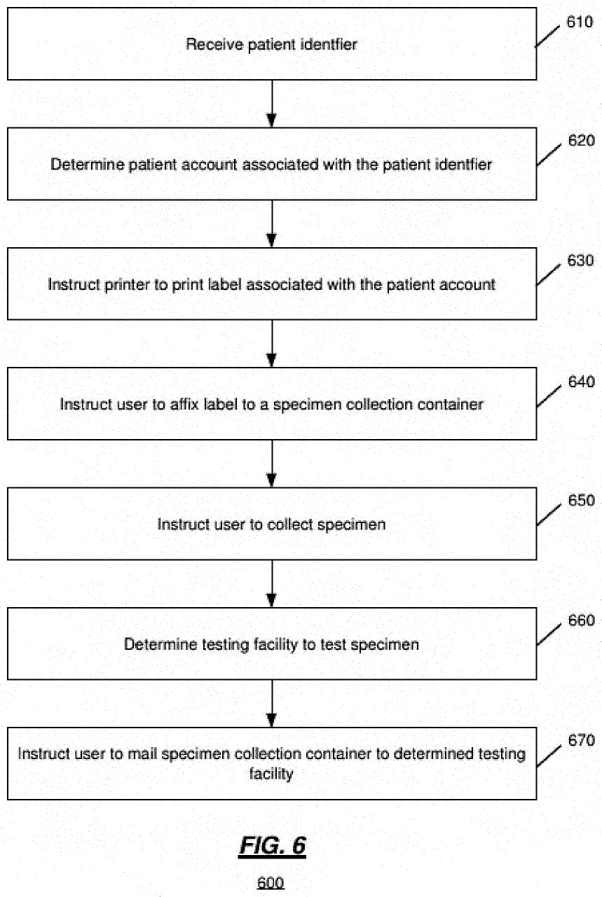

FIG. 6 is an illustration of a method 600 for collecting a specimen by a PSCT kit 110. The method 600 may be performed by one or more of the PSCT kit 110 and the laboratory server 120.

At 610, a patient identifier is received. The patient identifier 134 may the name of a patient 130 and may be extracted from an identification 137 associated with the patient 130 by the PSCT kit 110. In some embodiments, a scanner of the PSCT KIT 110 may scan a barcode on the identification 137 and may extract the patient identifier 134 from the barcode. Alternatively, the user of the PSCT kit 110 may manually enter the name of the patient 130 into a tablet computer associated with the PSCT kit 110.

At 620, a patient account associated with the patient identifier is determined. The patient account 125 may be determined by the PSCT kit 110. Alternatively, the PSCT kit 110 may provide the patient identifier 134 to the account engine 125 of the laboratory server 120 and the the account engine 125 may determine the patient account 135 that corresponds to the patient identifier 134. If no account exists, the PSCT kit 110 may collect information from the patient 130 to create a patient account 135.

At 630, the printer is instructed to print a label associated with the patient account. The instruction may be received by the printer from the tablet computer. The label may include a barcode that links the label to the patient account 135. The label may include additional information such as the name of the patient 130, an identifier of the particular test that is being performed, a current date and time, and the location of the patient 130. The label may be affixable to a specimen collection container 133.

At 640, the user is instructed to affix the label to a specimen collection container. The instruction may be displayed on the screen of the tablet computer.

At 650, the user is instructed to collect a specimen. The instruction may be displayed on the screen of the tablet computer. The specimen that is collected may depend on the particular test being performed. After collecting the specimen, the user may place the specimen in the labeled specimen collection container 113.

At 660, a testing facility to test the collected specimen is determined. The testing facility 150 may be determined by the PSCT kit 110 or may be determined by the laboratory server 120.

At 670, the user is instructed to mail the specimen collection container to the determined testing facility. The instruction may be displayed on the screen of the tablet computer. In some embodiments, the PSCT kit 110 may include one or more envelopes that may be used to mail the labeled specimen collection container 113.

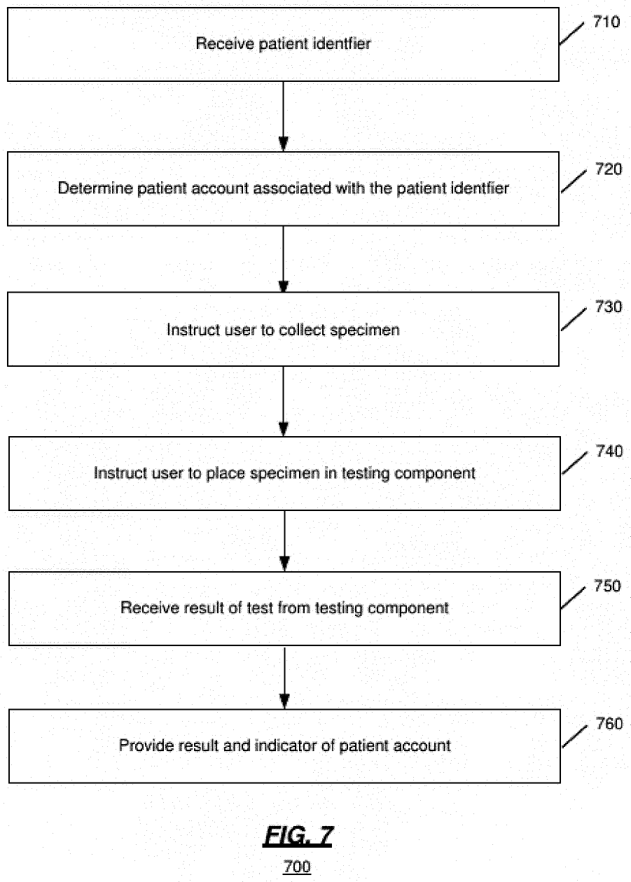

FIG. 7 is an illustration of a method 700 for testing a specimen by a PSCT kit 110. The method 700 may be performed by one or more of the PSCT kit 110 and the laboratory server 120.

At 710, a patient identifier is received. The patient identifier 134 may the name of a patient 130 and may be extracted from an identification 137 associated with the patient 130 by the PSCT kit 110.

At 720, a patient account associated with the patient identifier is determined. The patient account 125 may be determined by the PSCT kit 110. Alternatively, the PSCT kit 110 may provide the patient identifier 134 to the account engine 125 of the laboratory server 120 and the the account engine 125 may determine the patient account 135 that corresponds to the patient identifier 134. If no account exists, the PSCT kit 110 may collect information from the patient 130 to create a patient account 135.

At 730, the user is instructed to collect a specimen. The instruction may be displayed on the screen of the tablet computer. The specimen that is collected may depend on the particular test being performed.

At 740, the user is instructed to place the specimen in the testing component. The instruction may be displayed on the screen of the tablet computer. The testing component 313 may be a molecular analyzer.

At 750, results from the testing component are received. The results 115 may be received by the tablet computer from the molecular analyzer.

At 760, the results and indicator of the patient account are transmitted. The results 115 and the indicator of the patient account 135 of the patient 130 being tested may be transmitted to the account engine 125 of the laboratory server 120. The account engine 125 may store the results 115 and may associate the results 115 with the patient account 135. The results 115 may then be provided to the patient 130 by the report engine 129 in a report 119.

In some embodiments, even though the molecular analyzer tested the sample the PSCT kit 110 may further instruct the user to place the sample in the labeled specimen collection container 113, and to mail the container 113 to a testing facility 150 as described with respect to FIG. 6. The test performed by the testing facility 150 may verify the test performed by the molecular analyzer.

FIG. 8 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 8, an exemplary system for implementing aspects described herein includes a computing device, such as computing device 800. In its most basic configuration, computing device 800 typically includes at least one processing unit 802 and memory 804. Depending on the exact configuration and type of computing device, memory 804 may be volatile (such as random-access memory (RAM)), non-volatile (such as read-only memory (ROM), flash memory, etc.), or some combination of the two. This most basic configuration is illustrated in FIG. 8 by dashed line 806.

Computing device 800 may have additional features/functionality. For example, computing device 800 may include additional storage (removable and/or non-removable) including, but not limited to, magnetic or optical disks or tape. Such additional storage is illustrated in FIG. 8 by removable storage 808 and non-removable storage 810.

Computing device 800 typically includes a variety of computer readable media. Computer readable media can be any available media that can be accessed by the device 800 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 804, removable storage 808, and non-removable storage 810 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 600. Any such computer storage media may be part of computing device 800.

Computing device 900 may contain communication connection(s) 812 that allow the device to communicate with other devices. Computing device 800 may also have input device(s) 814 such as a keyboard, mouse, pen, voice input device, touch input device, etc. Output device(s) 816 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A portable specimen collection and testing kit comprising:
a case;
a printing component;
a computing component; and
a data entry component, wherein the computing component is adapted to:
receive a patient identifier from the data entry component;
determine a patient account associated with the patient identifier;
instruct the printing component to print a label with a computer-readable code that identifies the patient account and at least one test;
instruct a user of the portable specimen collection and testing kit to affix the label to a specimen collection container; and
instruct the user to place a specimen in the specimen collection container.

2. The portable specimen collection and testing kit of claim 1, wherein the computing component is further adapted to:
determine a testing facility to test the specimen; and
instruct the user to mail the specimen collection container to the determined testing facility.

3. The portable specimen collection and testing kit of claim 1, wherein the at least one test comprises one or more of a COVID-19 test, an Ebola test, a Zika test, a tuberculosis test, or a sexually transmitted disease test.

4. The portable specimen collection and testing kit of claim 1, wherein the computing component comprises a tablet computer and instructing the user of the portable specimen collection and testing kit to affix the label to the specimen collection container comprises displaying instructions to affix the label to the specimen collection container on a screen of the tablet computer.

5. The portable specimen collection and testing kit of claim 1, wherein the data entry component comprises a scanner.

6. The portable specimen collection and testing kit of claim 5, wherein the patient identifier is scanned from an identification associated with a patient.

7. The portable specimen collection and testing kit of claim 6, wherein the identification comprises a driver's license or a passport.

8. The portable specimen collection and testing kit of claim 1, further comprising a networking component that creates a network connection between the computing component and a cloud-based laboratory server.

9. The portable specimen collection and testing kit of claim 8, wherein the computing component adapted to determine the patient account associated with the patient identifier comprises the computing component adapted to:
provide the patient identifier to the cloud-based laboratory server via the network connection; and
receive the patient account from the cloud-based laboratory server via the network connection.

10. The portable specimen collection and testing kit of claim 8, wherein the networking component comprises a portable Wi-Fi hotspot.

11. A portable specimen collection and testing kit comprising:
a case;
a computing component;
a testing component; and
a data entry component, wherein the computing component is adapted to:
receive a patient identifier from the data entry component;
determine a patient account associated with the patient identifier;
instruct a user to place a specimen in the testing component;
receive a result of at least one test from the testing component; and
provide the result and indicator of the patient account.

12. The portable specimen collection and testing kit of claim 11, wherein the at least one test comprises one or more of a COVID-19 test, an Ebola test, a Zika test, a tuberculosis test, or a sexually transmitted disease test.

13. The portable specimen collection and testing kit of claim 11, wherein the data entry component comprises a scanner.

14. The portable specimen collection and testing kit of claim 13, wherein the identifier of the patient is scanned from an identification associated with a patient.

15. The portable specimen collection and testing kit of claim 14, wherein the identification comprises a driver's license or a passport.

16. The portable specimen collection and testing kit claim 11, further comprising a networking component that creates a network connection between the computing component and a cloud-based laboratory server.

17. The portable specimen collection and testing kit of claim 16, wherein the computing component adapted to determine the patient account associated with the patient identifier comprises the computing component adapted to:
provide the patient identifier to the cloud-based laboratory server via the network connection; and
receive the patient account from the cloud-based laboratory server via the network connection.

18. The portable specimen collection and testing kit of claim 16, wherein the computing component adapted to provide the result and indicator of the patient account comprises the computing account adapted to provide the result and indicator of the patient account to the cloud-based laboratory server through the network connection.

19. The portable specimen collection and testing kit of claim 16, wherein the networking component comprises a portable Wi-Fi hotspot.

20. The portable specimen collection and testing kit of claim 11, wherein the testing component comprises a molecular analyzer.
